(19) United States
(12) Patent Application Publication
(10) Pub. No.: US 2021/0290425 A1
(43) Pub. Date: Sep. 23, 2021

(54) ORTHOPEDIC SHOULDER SUPPORT FOR SHOULDER STABILIZATION

(71) Applicant: William Michael Cresse, Boise, ID (US)

(72) Inventor: William Michael Cresse, Boise, ID (US)

(21) Appl. No.: 16/823,190

(22) Filed: Mar. 18, 2020

(51) Int. Cl. A61F 5/37

(52) U.S. Cl. CPC A61F 5/3776; A61F 5/37

(57) ABSTRACT

Orthopedic shoulder supports and associated methods for producing the same are described herein. In various embodiments, the orthopedic shoulder support comprises a body having a recess configured to hold a user's arm and shoulder in a prescribed position. The recess is generally defined by a shoulder support surface, an arm support surface extending upwardly therefrom, and sidewalls extending upwardly from the shoulder support surface and arm support surface. In some embodiments, the orthopedic shoulder support further comprises a retention member configured to help retain the shoulder support in a position to hold the user's arm and shoulder in the prescribed position. In some embodiments, the dimensions of the recess are customized to the intended user. Details on these and various other aspects of the present technology are described herein.

### TECHNICAL FIELD

The present disclosure is generally directed to an orthopedic shoulder support for stabilizing a user's shoulder during sleep, and more specifically to an orthopedic pillow for holding a user's arm and shoulder in a prescribed position during recovery from shoulder injury or surgery and/or during periods of shoulder pain.

### BACKGROUND

The shoulder is one of the most movable joints in the body. It is also an unstable joint, in part because of the range of motion allowed. This instability increases the likelihood of joint injury, which can lead to a degenerative process where tissues break down and no longer function well. As a result, shoulder problems are one of the most common reasons for physician visits in the United States. Around 40% of Americans suffer from some level of shoulder pain, leading as many as 600,000 Americans to have shoulder surgery each year. However, their recovery times and outcomes are often limited by their inability to sleep post-operation, while stabilizing their shoulder for recovery.

Despite advances in medical operations and physical therapy available for shoulder pain, many patients are still confined to wearing a sling to bed during recovery periods. While slings can stabilize the shoulder joint, they are uncomfortable for the user, resulting in less sleep during an important stage of recovery. Consequently, this results in slower, less satisfying, and less complete recoveries. To remedy the discomfort patients experience with wearing slings to bed, some doctors prescribe opioid-based pain killers. While using opioid-based pain killers can result in better sleep, they can also interfere with the user's functioning during the day and can cause problems associated with addiction after recovery. Alternatively, some shoulder-comfort pillows allow users to position themselves for more comfort, but do not stabilize the shoulder nor prevent body weight from being carried by the shoulder. While comfort pillows can result in better sleep, the destabilization and loading of the shoulder joint can result in more daytime pain, reinjuries, and incomplete recoveries. Accordingly, there is a need for a device that can comfortably stabilize the shoulder joint to allow users to sleep during recovery periods without using potentially addictive medications and without risking reinjury or inhibiting recovery.

### BRIEF DESCRIPTION OF THE DRAWINGS

FIG. 1A is an isometric view of an orthopedic shoulder support configured in accordance with an embodiment of the present technology.

FIG. 1B is an isometric view of a user with their arm and shoulder being held in a prescribed position by the orthopedic shoulder support of FIG. 1A.

FIG. 2A is a side view illustrating a measurement of a user relevant to some embodiments of the present technology.

FIG. 2B is a top view illustrating two other measurements of a user relevant to some embodiments of the present technology.

FIG. 2C is an isometric view of the orthopedic shoulder support of FIG. 1A customized for a user according to the measurements illustrated in FIGS. 2A and 2B in accordance with an embodiment of the present technology.

FIG. 3 is an isometric view of an orthopedic shoulder support having a flexible retention strap configured in accordance with an embodiment of the present technology.

FIG. 4 is an isometric view of an orthopedic shoulder support having two retention members configured in accordance with another embodiment of the present technology.

FIG. 5 is an isometric view of an orthopedic shoulder support having a flexible retention strap configured in accordance with another embodiment of the present technology.

FIG. 6 is an isometric view of an orthopedic shoulder support configured in accordance with another embodiment of the present technology.

FIG. 7A is an isometric view of the orthopedic shoulder support of FIG. 6 in combination with a head support configured in accordance with an embodiment of the present technology.

FIG. 7B is an isometric view of a user using the orthopedic shoulder support and head support of FIG. 7A.

### DETAILED DESCRIPTION

The following disclosure describes various embodiments of orthopedic shoulder supports for comfortably stabilizing a shoulder joint of a user lying adjacent the orthopedic shoulder support. In some embodiments, the orthopedic shoulder support is comprised of a body having a recess. The recess is generally defined by a horizontal shoulder support surface, an arm support surface extending from the shoulder support surface at an upward angle, and vertical sidewalls extending upwardly from the shoulder support surface and the arm support surface.

The orthopedic shoulder support is configured to stabilize the shoulder joint by allowing a user's arm to rest on a comfortable surface while being held in a prescribed position (e.g., a position mimicking the position of an arm in a sling). When a user lies adjacent the orthopedic shoulder support, their shoulder rests on the shoulder support surface and their upper arm rests on the arm support surface. The sidewalls help to immobilize the arm and shoulder, thereby reducing the amount of undesired and/or unintentional movement through the shoulder joint.

In some embodiments, the orthopedic shoulder support can also include a retention member configured to help retain the orthopedic shoulder support in the proper position relative to the user. In some embodiments, the retention member can be made of a flexible material extending outwardly from a bottom surface of the orthopedic shoulder support. In use, the user lies on the retention member adjacent to the orthopedic shoulder support. In this way, the user's weight on the retention member restrains the orthopedic shoulder support from moving away from the user. In other embodiments, the retention member can be a flexible retaining strap secured to the orthopedic shoulder support and configured to be wrapped around a portion of the user's body to retain the orthopedic shoulder support in the proper position. In some embodiments, the retention member can be a flexible strap securely attached to the orthopedic shoulder support and configured to wrap around a portion of the user's arm. In this embodiment, the flexible retention strap holds the orthopedic shoulder support in the proper position while also restraining movement of the user's arm.

In some embodiments, the dimensions of various features of the orthopedic shoulder support can be customized to a particular user during manufacture. For example, the recess can be formed to fit the user's body (e.g., the length of the arm support surface can be approximately equal to the length of the user's upper arm). Such customization can improve the fit of the orthopedic shoulder support for the user, which is expected to improve the user's comfort and recovery. In other embodiments, the orthopedic shoulder support can be manufactured with additional features to enhance user comfort.

The terminology used below is to be interpreted in its broadest reasonable manner, even though it is being used in conjunction with a detailed description of certain examples of embodiments of the present technology. Indeed, certain terms may even be emphasized below; however, any terminology intended to be interpreted in any restricted manner will be specifically defined as such in this Detailed Description section. For ease of reference, the orthopedic shoulder support may be described herein with reference to top and bottom, head and foot, upwards and downwards, and/or lateral, horizontal, or vertical relative to the spatial orientation of the embodiments shown in the figures. It is to be understood that the orthopedic shoulder support, however, can be moved to, and used in, different spatial orientations without changing the structure and/or function of the disclosed embodiments of the present technology.

The figures have not necessarily been drawn to scale. For example, the dimensions of some of the elements in the figures may be expanded or reduced to help improve the understanding of the embodiments. Moreover, while the disclosed technology is amenable to various modifications and alternative forms, specific embodiments have been shown by way of example in the figures and are described in detail below. The intention, however, is not to limit the embodiments described. On the contrary, the embodiments are intended to cover all modifications, equivalents, and alternatives falling within the scope of the disclosed technology. Component details may be abstracted in the figures to exclude details such as position of components and certain precise connections between such components when such details are unnecessary for a complete understanding of how to make and use the invention.

The orthopedic shoulder supports are sometimes described herein in the context of an orthopedic pillow configured to help stabilize a shoulder joint while the user rests adjacent to the pillow. It will be appreciated, however, that the orthopedic shoulder support can be made from relatively firm materials such as plastics, air pillows, rubber, closed cell foams, rigid frames with mesh or netting defining the shoulder support surface and/or arm support surface, and/or other suitable materials.

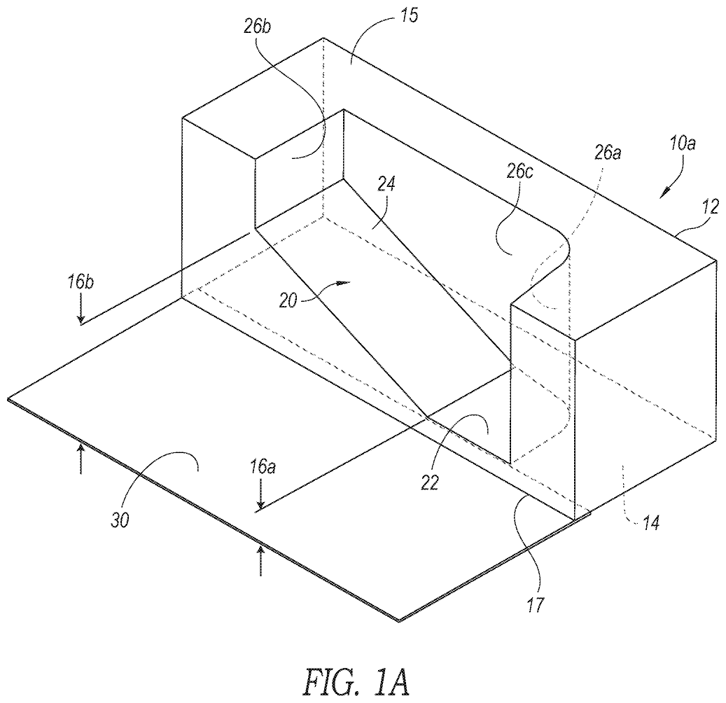

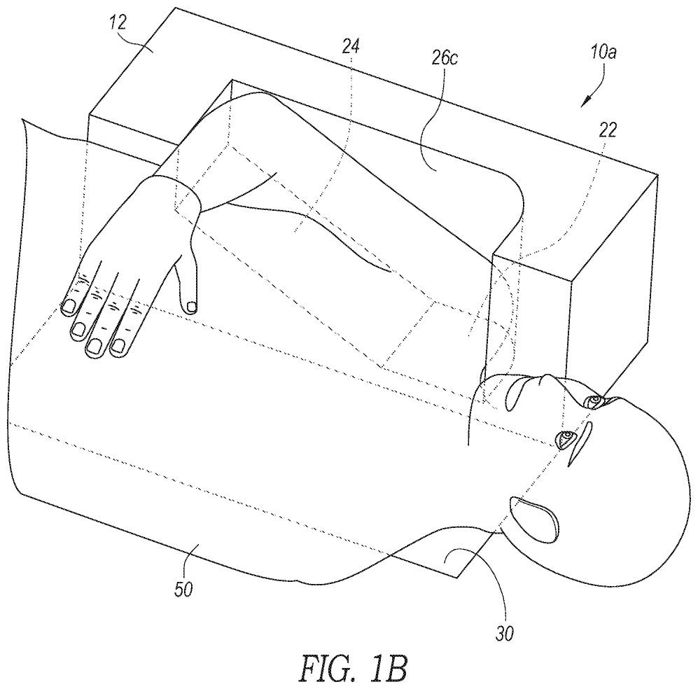

FIG. 1A is an isometric view of an orthopedic shoulder support 10a configured in accordance with an embodiment of the present technology for stabilizing a user's arm and shoulder, and FIG. 1B is an isometric view of a user with their arm and shoulder being held in a prescribed position by the orthopedic shoulder support 10a. Referring first to FIG. 1A, the orthopedic shoulder support 10a has a body 12 with a bottom surface 14 and a recess 20. The recess 20 is generally defined by a shoulder support surface 22, an arm support surface 24, and sidewalls 26a-c extending upwardly therefrom. In the illustrated embodiment, the orthopedic shoulder support 10a also includes a retention member 30 extending outwardly from an edge 17 of the bottom surface 14.

In the illustrated embodiment, the shoulder support surface 22 is a generally horizontal planar surface that is positioned at a first distance or height 16a above the bottom surface 14 of the body 12. The arm support surface 24 extends from the shoulder support surface 22 at an upward angle relative to the bottom surface 14 and transitions from the first height 16a at a proximal end portion to a second, greater height 16b at a distal end portion. In the illustrated embodiment, the sidewalls 26a-c extend vertically, or generally vertically, from the edges of the shoulder support surface 22 and the arm support surface 24 and include: a first sidewall 26a adjacent the shoulder support surface 22, a second sidewall 26b adjacent the arm support surface 24, and a third sidewall 26c adjacent the shoulder support surface 22 and the arm support surface 24 and extending from the first sidewall 26a to the second sidewall 26b. In other embodiments, the recess 20 can have sidewalls extending on only two sides of the shoulder support surface 22 and the arm support surface 24. For example, in such an embodiment, the distal end portion of the arm support surface 24 can extend to a top surface 15 of the body 12, thereby omitting the second sidewall 26b.

Referring next to FIG. 1B, when a user 50 lies adjacent the orthopedic shoulder support 10a, the recess 20 can hold the user's arm and shoulder in a prescribed position. That is, the orthopedic shoulder support 10a holds the user's arm and shoulder in a position that is the same as, or at least similar to, the position an arm sling would hold the user's arm and shoulder. For example, as shown in FIG. 1B, when the user lies adjacent the orthopedic shoulder support 10a, the user's shoulder rests on the shoulder support surface 22 and the user's arm rests on the arm support surface 24. Further, when the user lies in this position, the sidewalls 26a-c help constrain and reduce undesirable and/or unintentional movement of the user's arm by constraining the directions in which the user's arm can move.

In some embodiments, the retention member 30 is a relatively thin sheet of flexible material (e.g., a textile material such as a fabric or a cloth (e.g., canvas); rubber, such as latex or neoprene; plastic; foam such as polyurethane foam or Serene® polyfoam; etc.) extending outwardly from the bottom surface 14 of the body 12. In some embodiments, the retention member 30 has a thickness of from about 0.5 to about 30 millimeters (mm), from about 1 to about 10 mm, or about 2 mm. In some embodiments, retention member 30 can be attached to the bottom surface 14 of the body 12 by an adhesive (e.g., 3M®'s Foam Fast 74 Spray Adhesive, 3M®'s HI-STRENGTH 90 Contact Adhesive, National Guards' 1633 Foam Lock, etc.). In other embodiments, the retention member can be attached to the body 12 by various other means such as fasteners, incorporation into a cover for the orthopedic shoulder support, hook and loop fasteners (e.g., Velcro®), sewing, high temperature bonding (e.g., heat staking), etc. In the illustrated embodiment, the retention member 30 is configured to be positioned underneath the user when the user lies adjacent to the orthopedic shoulder support 10a as shown in FIG. 1B. When the user rests on the retention member 30, the user's weight restrains the retention member 30, which in turn prevents the orthopedic shoulder support 10a from moving away from the user, thereby maintaining the orthopedic shoulder support 10a in the proper position to hold the user's arm and shoulder. In the illustrated embodiment, the retention member 30 extends outwardly from a longitudinal edge 17 of the bottom surface 14. In other embodiments, the flexible material can extend outwardly from only a portion of the edge 17.

In the illustrated embodiment, the retention member 30 is secured to a portion of the bottom surface 14 of the body 12. In various embodiments, the retention member can be secured to greater or smaller portions of the bottom surface 14. For example, in some embodiments the retention member 30 can be secured only to the longitudinal edge 17 of the bottom surface 14. In other embodiments, the retention member can be secured to the entire bottom surface 14. Further, in some embodiments, the retention member has a width of from about 10 to about 90 centimeters (cm), from about 30 to about 70 cm, or about 45 cm. As a further example, in an embodiment where the retention member 30 has a width of about 45 cm, about 20 cm of the width of the retention member 30 can be attached to the bottom surface 14 while about 25 cm of the width of the retention member 30 extends outwardly from the longitudinal edge 17 of the bottom surface 14.

In some embodiments, the orthopedic shoulder support can be formed from a compressible material. For example, in some embodiments, the orthopedic shoulder support can be formed from a foam, such as an open cell polyurethane foam having a density of from about 0.5 to about 3.5 pounds per cubic foot (lbs/ft³), from about 1.5 to about 2.5 lbs/ft³, or about 1.8 lbs/ft³, and/or a foam having an Indentation Load Deflection (ILD) of from about 50 to about 200, from about 80 to about 150, or about 100. In other embodiments, the orthopedic shoulder support can be formed from other types of foam (e.g., closed cell foam) having other densities and/or ILDs. In further embodiments, the body 12 can be formed from other materials (e.g., pliable plastics; air pillow(s); rubber, such as latex or neoprene; fabric bags stuffed with foam mulch, down, or batting; rigid frames with mesh or netting for the shoulder support surface and/or arm support surface; etc.).

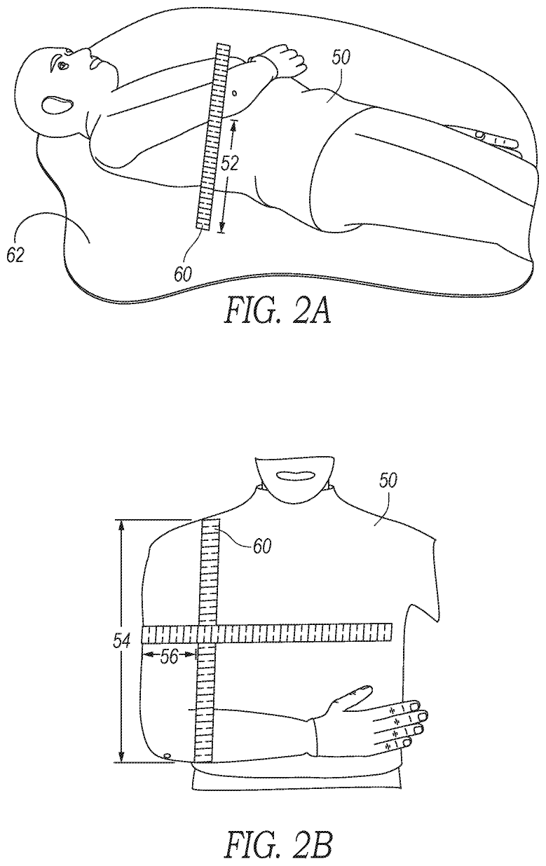

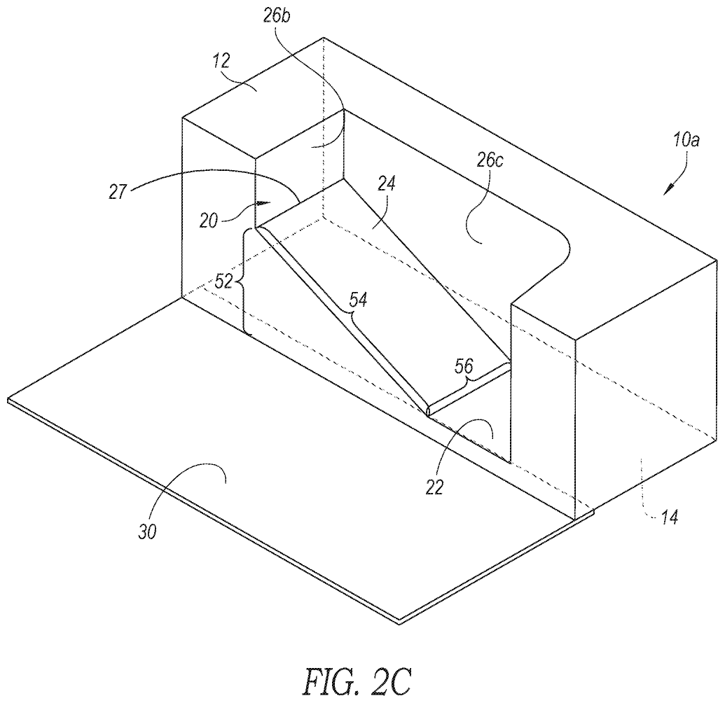

FIG. 2A is a side view illustrating a measurement of a user 50 relevant to some embodiments of the present technology. In the illustrated embodiment, a height 52 corresponds to the height of the user's stomach and/or chest above a support surface 62 the user 50 is laying on. The height 52 is approximately equal to the resting elevation of the user's lower arm when the user 50 lies in the prescribed position. The height 52 can be determined using a measuring instrument 60, such as a ruler, tape measure, tailors' tape; or a three-dimensional (3D) laser scanner, structured white-light light emitting diode (LED) scanner, structured blue-light LED scanner, and/or an application on a smartphone.

FIG. 2B is a top view illustrating two other measurements of the user 50 relevant to some embodiments of the present technology. In the illustrated embodiment, a length 54 is approximately equal to the length of the user's upper arm, and a width 56 is approximately equal to the maximum width of the user's upper arm and/or shoulder. The length 54 and the width 56 can also be determined using the measuring instrument 60.

FIG. 2C is an isometric view of the orthopedic shoulder support 10a of FIG. 1A customized for a user according to the measurements illustrated in FIGS. 2A and 2B in accordance with an embodiment of the present technology. In the illustrated embodiment, the recess 20 is at least partly defined by the measurements illustrated in FIGS. 2A and 2B. For example, a distal edge 27 of the arm support surface 24 can be positioned at the height 52 (FIG. 2A) above the bottom surface 14 of the body 12; the length of the arm support surface 24 can be equal to the length 54 (FIG. 2B); and the width of the arm support surface 24 can be equal to the width 56 (FIG. 2B). In some embodiments, the shoulder support surface 22 and the arm support surface 24 have the same width, such that the width of the shoulder support surface 22 is also equal to the width 56.

In some embodiments, for example, the orthopedic shoulder support 10a can be customized such that the height 52 of the distal edge 27 of the arm support surface 24 can be from about 17 to about 28 cm; the length 54 of the arm support surface 24 can be from about 30 to about 44 cm; and/or the width 56 of the arm support surface 24 can be from about 7 to about 16 cm. In some embodiments, a length of the shoulder support surface 22 can be from about 7 to about 23 cm, and/or a width of the shoulder support surface 22 can be from about 7 to about 16 cm (e.g., approximately equal to the width 56 of the arm support surface 24). Further, in some embodiments, the orthopedic shoulder support 10a can be customized such that the height of the shoulder support surface 22 (i.e., the height 16a above the bottom surface 14 (FIG. 1A)) can be from about 0.5 to about 5 cm, from about 1 to about 3 cm, or about 1.3 cm.

In some embodiments, the orthopedic shoulder support 10a can be customized to a range of sizes. For example, the orthopedic shoulder support 10a can have an XS, S, M, L, and/or XL size with dimensions in the ranges given above. In some embodiments, for example, a size XS orthopedic shoulder support 10a can be sized such that the distal edge 27 of the arm support surface 24 is at a height 52 of about 17 cm; the arm support surface 24 has a length of about 30 cm; the arm support surface 24 has a width of about 7 cm; the shoulder support surface 22 has a length of about 7 cm; and/or the shoulder support surface has a width of about 7 cm. In such embodiments, the dimensions of features of the recess 20 can step up between sizes in equal increments or in unequal increments. For example, in some embodiments, the length of the arm support surface 24 can change by increments of from about 1.5 to about 4.0 cm, or about 2.5 cm between sizes.

Customizing the dimensions of the orthopedic shoulder support 10a to be equal to or at least approximately equal to the measurements of a user as described above is expected to significantly increase the comfort of the orthopedic shoulder support 10a. Customizing the fit of the orthopedic support 10a is also expected to improve the stabilizing function of the orthopedic shoulder support 10a by limiting the user's arm movement.

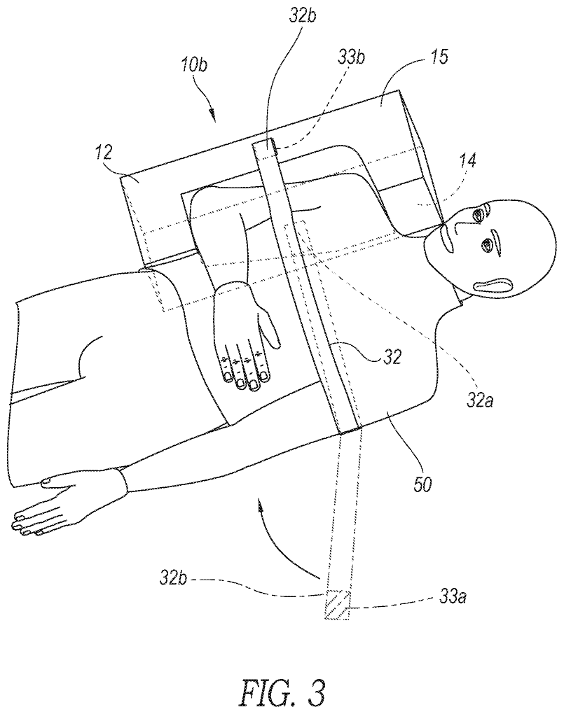

FIG. 3 is an isometric view of an orthopedic shoulder support 10b configured in accordance with another embodiment of the present technology. In the illustrated embodiment, the orthopedic shoulder support 10b is at least generally similar in structure and function to the orthopedic shoulder support 10a described in detail above with reference to FIG. 1A. For example, the orthopedic shoulder support 10b includes the body 12 having the recess 20 defined by the shoulder support surface 22, the arm support surface 24, and the sidewalls 26a-c (FIG. 1A). In the illustrated embodiment, however, the orthopedic shoulder support 10b further includes a flexible retention strap 32 (e.g., in place of the retention member 30 (FIG. 1A)).

In the illustrated embodiment, the flexible retention strap 32 has a first end portion 32a and a second end portion 32b. The first end portion 32a can be fixedly attached (e.g., by an adhesive) to the bottom surface 14 of the body 12, and the second end portion 32b can be releasably attached to the top surface 15 of the body 12 by fasteners 33a and 33b. In some embodiments, the fastener 33a can be one portion (e.g., a hook portion) of a hook and loop fastener system (e.g., Velcro®) and the fastener 33b can be the other portion (e.g., a loop portion) of the hook and loop fastener. In other embodiments, the second end portion 32b can be releasably attached to the body 12 by various other fasteners (e.g., a buckle, a clip fastener, an adhesive, etc.) In some embodiments, both the first end portion 32a and the second end portion 32b can be releasably attached to the body 12. In some embodiments, the first end portion 32a and the second end portion 32b can be attached to various other surfaces of the body 12 (e.g., the first end portion 32a can be attached to the backside of the body 12). In some embodiments, the first end portion 32a and the second end portion 32b can be attached to the body 12 in the same place and/or each other.

By wrapping around the user's body, the flexible retention strap 32 holds the orthopedic shoulder support 10b against the user's body, and thereby holds the orthopedic shoulder support 10b in a proper position to support the user's arm and shoulder. It will be understood that while the flexible retention strap 32 is illustrated wrapping around the user's torso and free arm, in other embodiments the flexible retention strap 32 can wrap around only the user's torso to accomplish the same function.

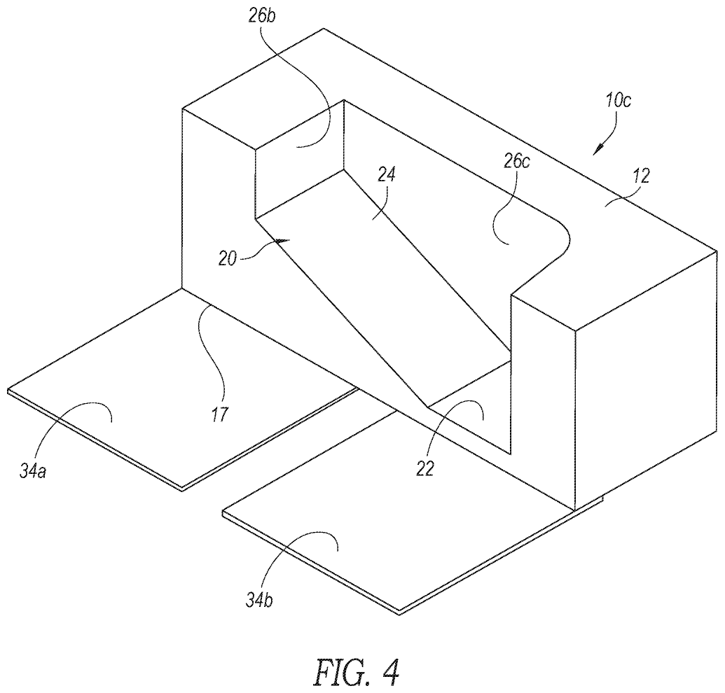

FIG. 4 is an isometric view of an orthopedic shoulder support 10c configured in accordance with another embodiment of the present technology. In the illustrated embodiment, the orthopedic shoulder support 10c is at least generally similar in structure and function to the orthopedic shoulder support 10a described in detail above with reference to FIG. 1A. For example, the orthopedic shoulder support 10c includes the body 12 having the recess 20 defined by the shoulder support surface 22, the arm support surface 24, and the sidewalls 26a-c. In the illustrated embodiment, however, the retention member 30 (FIG. 1A) is split into a first retention member 34a and a second retention member 34b extending outwardly from the longitudinal edge 17 of the body 12. As discussed in detail above, the first and second retention members 34a, 34b can be comprised of a flexible material configured to be positioned underneath a user, such that the weight of the user restrains the retention members 34a, 34b and keeps the orthopedic shoulder support 10c from moving away from the user.

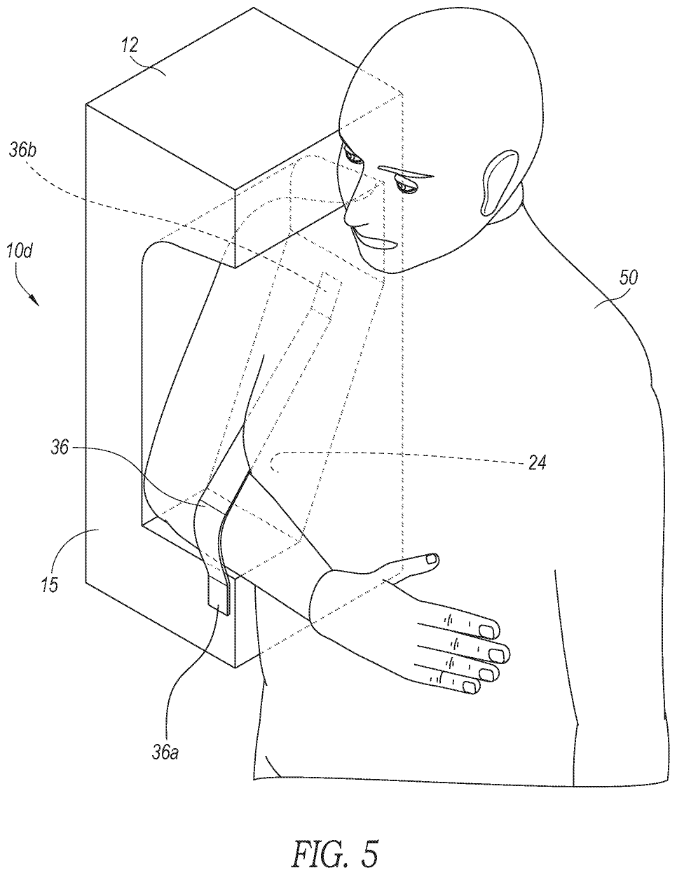

FIG. 5 is an isometric view of an orthopedic shoulder support 10d configured in accordance with a further embodiment of the present technology. In the illustrated embodiment, the orthopedic shoulder support 10d is at least generally similar in structure and function to the orthopedic shoulder support 10a described in detail above with reference to FIG. 1A. For example, the orthopedic shoulder support 10d includes the body 12 having the recess 20 defined by the shoulder support surface 22, the arm support surface 24, and the sidewalls 26a-c (FIG. 1A). In the illustrated embodiment, however, the orthopedic shoulder support 10d further includes a flexible retention strap 36 (e.g., in place of the retention member 30 (FIG. 1A)).

In the illustrated embodiment, the flexible retention strap 36 has a first end portion 36a and a second end portion 36b. The first end portion 36a can be fixedly attached to the top surface 15 of the body 12 (e.g., by adhesives) and the second end portion 36b can be releasably attached to the arm support surface 24 (e.g., using hook and loop fasteners (not shown)). In some such embodiments, the user 50 or another person can guide the second end portion 36b between the user's arm and torso to attach to the arm support surface. In some embodiments, both the first end portion 36a and the second end portion 36b can be releasably attached (e.g., using hook and loop fasteners) to the top surface 15 of the body 12 and the arm support surface 24, respectively. In some embodiments, both the first end portion 36a and the second end portion 36b can be fixedly attached (e.g., by an adhesive) to the top surface 15 of the body and the arm support surface 24, respectively. In some such embodiments, the user's shoulder can be placed within the recess 20 (FIG. 4) of the body 12 and the user's wrist and forearm can be passed under the strap 36. In some embodiments, the first end portion 36a and the second end portion 36b can be attached to various other portions of the body 12. For example, in some embodiments, the first end portion 36a can be attached to the bottom surface 14 (FIG. 1A) of the body 12 using an adhesive, the flexible retention strap 36 can wrap around the user's arm, and the second end portion 36b can be attached to the top surface 15 of the body 12 using hook and loop fasteners (e.g., similar to the orientation of the flexible retention strap 32 (FIG. 3), except the flexible retention strap 36 only wraps around the user's arm).

In use, the flexible retention strap 36 retains the orthopedic shoulder support 10d in the proper position to support the user's arm and shoulder (e.g., in a manner similar to the flexible retention strap 32 discussed above with respect to FIG. 3). In addition, by wrapping around the user's arm, the flexible retention strap 36 restrains movement of the user's arm, particularly in the vertical direction. That is, in use, the flexible retention strap 36 can simultaneously help retain the orthopedic shoulder support 10d in the proper position to support the user's arm and shoulder and restrain unintentional movement of the user's arm.

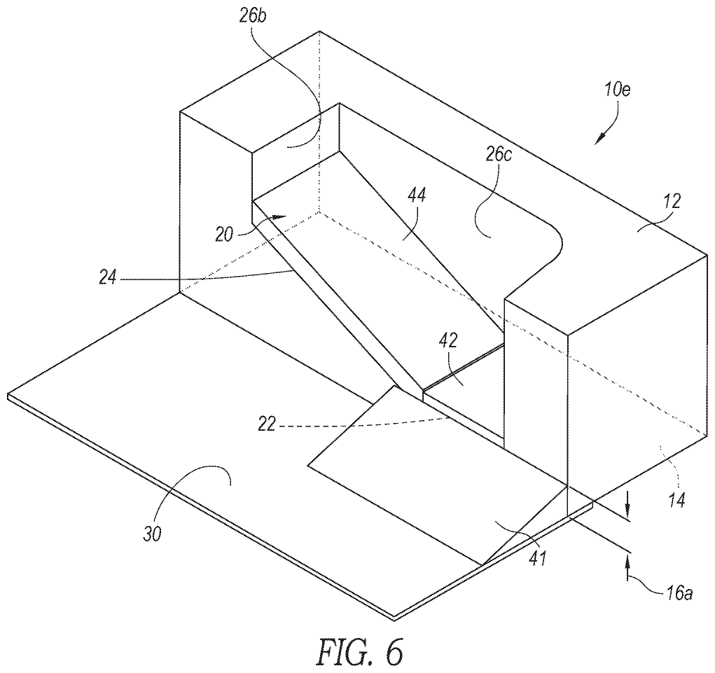

FIG. 6 is an isometric view of an orthopedic shoulder support 10e configured in accordance with another embodiment of the present technology. In the illustrated embodiment, the orthopedic shoulder support 10e is at least generally similar in structure and function to the orthopedic shoulder support 10a described in detail above with reference to FIG. 1A. For example, the orthopedic shoulder support 10d includes the body 12 having the recess 20 defined by the shoulder support surface 22, the arm support surface 24, and the sidewalls 26a-c (FIG. 1A). In the illustrated embodiment, however, the orthopedic shoulder support 10e further includes a shoulder transition pad 41, a shoulder support surface pad 42, and an arm support surface pad 44.

The shoulder transition pad 41 extends outwardly from the body 12 from a position adjacent the shoulder support surface 22. In the illustrated embodiment, the shoulder transition pad 41 extends laterally outwards at a right angle, or about a right angle, relative to the shoulder support surface 22. Further, the shoulder transition pad 41 extends outwardly at a downward slope. For example, in some embodiments, the shoulder transition pad 41 extends outwardly at a downward slope from the first height 16a adjacent to the shoulder support surface 22 to the base surface 14 at a distal end of the shoulder transition pad 41. In other embodiments, the shoulder transition pad 41 extends outwardly at a downward slope from different heights. For example, in some embodiments the shoulder transition pad 41 extends outwardly at a downward slope from about a top surface of the shoulder support surface 22 to a height (not shown) above the base surface 14.

In some embodiments, the shoulder transition pad 41 can be attached to the body 12 by an adhesive. In other embodiments, the shoulder transition pad 41 can be attached to the body 12 by fastener mechanisms (e.g., hook and loop fasteners). In other embodiments, the shoulder transition pad 41 can be manufactured as an integral element of the unitary body 12. In some embodiments, the shoulder transition pad 41 can be made from a comfort improving material (e.g., a foam such as an open cell polyurethane foam or gel infused foam, microbeads, down, etc.). For example, in some embodiments, the shoulder transition pad 41 can be made from an open cell polyurethane foam having a density of from about 1 to about 5 lbs/ft³, from about 2 to about 4 lbs/ft³, or about 2.5 lbs/ft³, and/or an ILD of from about 5 to about 50, from about 15 to about 40, or about 24. In some embodiments, the shoulder transition pad 41 can be made from multiple materials. For example, the structure of the shoulder transition pad 41 can be made from a relatively stiff material (e.g., a foam with a relatively high ILD) having an upper surface coated with a comfort improving material (e.g., a foam with a relatively low ILD).

When a user lies adjacent to the orthopedic shoulder support 10e, the shoulder transition pad 41 provides a support surface that transitions the user's shoulder into the shoulder support surface 22. The transitional support surface is expected to increase the user's comfort while using the orthopedic shoulder support 10e.

The shoulder support surface pad 42 is disposed on the shoulder support surface 22 and the arm support surface pad 44 is disposed on the arm support surface 24. In the illustrated embodiment, the shoulder support surface pad 42 is disposed over the entire surface of the shoulder support surface 22. In other embodiments, the shoulder support surface pad 42 can be disposed over only a portion of the shoulder support surface 22 (e.g., covering only a center portion of the shoulder support surface 22). Similarly, in the illustrated embodiment, the arm support surface pad 44 is disposed over the entire surface of the arm support surface 24. In other embodiments, the arm support surface pad 44 can be disposed over only a portion of the arm support surface 24.

In the illustrated embodiment, the shoulder support surface pad 42 and the arm support surface pad 44 are relatively thin pads with the same thickness. For example, the shoulder support surface pad and/or the arm support surface pad 44 can have a thickness of from about 1 to about 8 cm, from about 3 to about 6 cm, or about 5.1 cm. In other embodiments, the shoulder support surface pad 42 and arm support surface pad 44 can have thicknesses different from each other. For example, in some embodiments, the shoulder support surface pad 42 can have a thickness of about 4 cm and the arm support surface pad 44 can have a thick of about 3 cm.

In some embodiments, the shoulder support surface pad 42 and arm support surface pad 44 can be attached to the body 12 by an adhesive and/or various types of fasteners (e.g., hook and loop fasteners). In other embodiments, the shoulder support surface pad 42 and the arm support surface pad 44 can be simply placed on the shoulder support surface 22 and the arm support surface 24 respectively. In such embodiments, the user's weight on the support surface pads 42 and 44 can retain the support surface pads 42 and 44 in position.

The shoulder support surface pad 42 and arm support surface pad 44 can be made from comfort improving materials. For example, the shoulder support surface pad 42 and arm support surface pad 44 can be made from a relatively soft foam. In some embodiments, the shoulder support surface pad 42 and arm support surface pad 44 can be an open cell polyurethane foam having a density from about 1 to about 5 lbs/ft³, from about 2 to about 4 lbs/ft³, or about 2.5 lbs/ft³, and/or an ILD from about 5 to about 50, from about 15 to about 40, or about 24. In other embodiments, the shoulder support surface pad 42 and arm support surface pad 44 can be made from other materials (e.g., other foams, gel, microbeads, down, cotton) configured to improve the user's comfort while resting their arm and shoulder on the shoulder support surface pad 42 and arm support surface pad 44.

In some embodiments, the orthopedic shoulder support 10e can include a pad on various other surfaces of the body 12 in addition to the shoulder support surface pad 42 and the arm support surface pad 44. For example, in some embodiments, the orthopedic shoulder support 10e can further include a pad on the sidewalls 26a-c, the top surface 15 (FIG. 1A), and/or any other surface the user's skin may contact. In such embodiments, the additional pads can also be made from comfort improving materials (e.g., relatively soft foams, gels, microbeads, down, cotton, etc.) to improve the user's comfort while using the orthopedic shoulder support 10e.

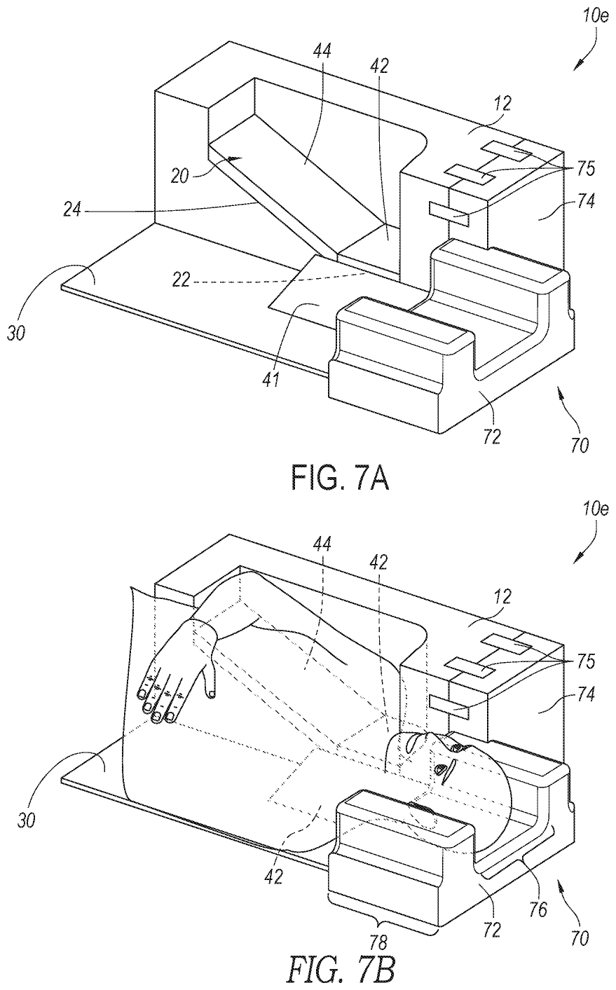

FIG. 7A is an isometric view of the orthopedic shoulder support 10e of FIG. 6 in combination with a head support 70 configured in accordance with another embodiment of the present technology. In the illustrated embodiment, the head support 70 includes a head supporting member 72 and an attachment member 74. In the illustrated embodiment, the attachment member 74 is connected to the body 12 by patches 75 that can include fastener mechanisms, such as hook and loop fastener mechanisms, adhesive tape, buckles, and/or other means of attachment. In other embodiments, the patches 75 can be disposed between the body 12 and the attachment member 74, the attachment member 74 can be attached to the body 12 by an adhesive, or the attachment member 74 can be formed as an integral portion of the body 12.

In the illustrated embodiment, the attachment member 74 connects the head supporting member 72 to the body 12. In the illustrated embodiment, the head supporting member 72 has an upward-facing u-shape configured to receive and support and/or stabilize a user's head when using the orthopedic shoulder support 10e. In other embodiments, the head supporting member 72 can have various other suitable shapes configured to support and/or stabilize a user's head (e.g., elliptical, flat, etc.). In some embodiments, the head supporting member 72 is a block of shape-conforming foam (e.g., Serene® polyfoam, or other shape-conforming foams) configured to generally conform to the shape of a user's head and hold the head vertical. In the illustrated embodiment, the head supporting member 72 is integrally connected to the attachment member 74. In other embodiments, the head supporting member 72 and attachment member 74 can be individual pieces attached by an adhesive.

FIG. 7B is an isometric view of a user 50 using the orthopedic shoulder support 10e and head support 70 of FIG. 7A in accordance with another embodiment of the present technology. In some embodiments, the head support 70 can be customized for the user 50. For example, the head supporting member 72 has a length 78 equal, or at least approximately equal, to the length of the user's head and an interior width 76 equal, or at least approximately equal, to the maximum width of the user's head. In some embodiments, for example, the head supporting member 72 can have a length 78 of from about 17 to about 26 cm, and/or a width 76 of from about 12 to about 18 cm. In some embodiments, the user's head can be measured with a measuring instrument 60 (FIGS. 2A-B), such as a ruler, tape measure, tailors' tape; or a 3D laser scanner, structured white-light LED scanner, structured blue-light LED scanner, and/or an application on a smartphone.

In some embodiments, the length 78 and the interior width 76 can be customized in addition to the customized features discussed above with respect to FIGS. 2A-2C. In other embodiments, some subset of the features discussed above can be customized to the user 50. For example, in one embodiment, the arm supporting surface 24 can have a second distance equal to the height 52 customized to the user (FIGS. 2A and 2C) and the head supporting member 72 can have an interior width equal to the width 76 customized to the user. The customization of any and/or all of the features discussed above improves the fit of the orthopedic shoulder support 10e and is thereby expected to increase user comfort. The improved fit is also expected to increase the stabilization of the shoulder. Both improvements are expected to lead to fuller, faster recoveries for users.

The orthopedic shoulder support of any of the embodiments discussed above can be manufactured using various approaches known in the art. In some embodiments, the orthopedic shoulder support can be manufactured by forming a generally horizontal shoulder support surface positioned at a first distance above a bottom surface of the orthopedic shoulder support; forming an arm support surface extending from the shoulder support surface at an upward angle relative to the bottom surface, the arm support surface having a proximal end portion positioned at the first distance above the bottom surface and a distal end portion positioned at a second distance above the bottom surface, the second distance being greater than the first distance; and forming at least one sidewall extending upwardly from the shoulder support surface and the arm support surface. In such embodiments, the shoulder support surface, the arm support surface, and the at least one sidewall define a recess in a body of the orthopedic shoulder support.

In some embodiments, for example, the orthopedic shoulder support can be manufactured as a unitary member, in which the recess is formed by removing material from a block of raw material (e.g., by cutting the block of raw material) to form the features of the recess discussed above. In some embodiments, the block of raw material is a pre-manufactured body of the orthopedic shoulder support. In some embodiments, the body is also formed by removing material from a block of raw material to define the body. In other embodiments, the orthopedic shoulder support can be manufactured in two or more individual pieces that are then attached together, e.g. by adhesive bonding. For example, the arm support surface, shoulder support surface, and/or sidewalls can be manufactured individually and can then be attached together (e.g., by adhesive bonding) to form the orthopedic shoulder support.

In some embodiments, the manufacturing process can create an orthopedic shoulder support that is customized to the intended user (e.g., a post-surgery patient). In these embodiments, measurements are taken of the user and used to tailor the features of the recess and/or head support.

The measurements can include, for example, a first measurement of the length of the user's shoulder from a top of the user's shoulder to a bottom of the user's shoulder; a second measurement of the length of the user's upper arm from the user's shoulder to a distal portion of the user's elbow; a third measurement of the resting height of the user's arm from the user's back to the user's elbow while the user is in the prescribed position; and/or a fourth measurement of a maximum width of the user's upper arm. For example, a measurement of a typical user might result in a first measurement (of the length of the user's shoulder) of from about 7 to about 16 cm; a second measurement (of the length of the user's upper arm) of from about 30 to about 44 cm; a third measurement (of the resting height of the user's arm) of from about 17 to about 28 cm; and/or a fourth measurement (of the width of the user's upper arm) of from about 7 to about 16 cm.

The first, second, third, and fourth measurements can be used to customize the recess to the user. In some embodiments, the shoulder support surface has a length of about the first measurement and/or a depth of about the fourth measurement. In some embodiments, the arm support surface has a length of about the second measurement, a depth of about the fourth measurement, and/or a second distance of about the third measurement.

In some embodiments, the measurements of the user can also include a fifth measurement of the length of the user's head, from the top of the user's head to the bottom of the user's head; and/or a sixth measurement of the maximum width of the user's head. The fifth and sixth measurements can be used to customize the head supporting member to the user. In some embodiments, the head supporting member has a length of about the fifth measurement and/or an interior width of about the sixth measurement.

It will be appreciated that the manufacturing process can customize the orthopedic shoulder support and/or head supporting member using various subsets of the measurements discussed above.

From the foregoing, it will be appreciated that specific embodiments of the invention have been described herein for purposes of illustration, but that various modifications may be made without deviating from the scope of the invention. Further, while various advantages associated with certain embodiments of the invention have been described above in the context of those embodiments, other embodiments may also exhibit such advantages, and not all embodiments need necessarily exhibit such advantages to fall within the scope of the invention. Accordingly, the invention is not limited except as by the appended claims.

We/I claim:

1. An orthopedic shoulder support comprising:
a body, wherein the body includes a bottom surface configured to contact a support surface on which the body is placed, wherein the body further includes a recess configured to hold a user's arm and shoulder in a prescribed position when the user lies adjacent to the shoulder support, and wherein the recess is defined by—
a generally horizontal shoulder support surface positioned at a first distance above the bottom surface;
an arm support surface extending from the shoulder support surface at an upward angle relative to the bottom surface, the arm support surface having a proximal end portion positioned at the first distance above the bottom surface and a distal end portion positioned at a second distance above the bottom surface, the second distance being greater than the first distance; and
a generally vertical sidewall extending upwardly adjacent to the shoulder support surface and the arm support surface.

2. The orthopedic shoulder support of claim 1, further comprising a retention member extending outwardly from the body adjacent the bottom surface and configured to support the user when the user lies adjacent to the orthopedic shoulder support.

3. The orthopedic shoulder support of claim 1, wherein:
the shoulder support surface has a first width that is at least approximately equal a maximum width of the user's arm;
the arm support surface has a second width that is at least approximately equal to the first width; and
the arm support surface has a length that is at least approximately equal to a length from the user's shoulder to a user's elbow.

4. The orthopedic shoulder support of claim 3 wherein the second distance is at least approximately equal to a distance from the support surface to a user's stomach when the user lies on the support surface.

5. The orthopedic shoulder support of claim 1 wherein the body further comprises a shoulder transition pad extending outwardly from the shoulder support surface at a downward angle relative to the bottom surface, wherein the arm support surface extends from the shoulder support surface in a first direction, and wherein the shoulder transition pad extends from the shoulder support surface in a second direction, the second direction being at about a right angle to the first direction.

6. The orthopedic shoulder support of claim 1 further comprising:
a shoulder support surface pad disposed on the shoulder support surface; and
an arm support surface pad disposed on the arm support surface.

7. The orthopedic shoulder support of claim 1, further comprising at least one flexible retention strap secured to a first side of the body and configured to wrap around a user and be releasably attached to a second side of the body.

8. The orthopedic shoulder support of claim 1, further comprising at least one flexible retention strap secured to a first side of the body near a distal end of the arm support surface and configured to wrap around a user's arm and be releasably attached to second side of the body near a proximal end of the arm support surface.

9. The orthopedic shoulder support of claim 1 further comprising a head supporting portion attached to the body, wherein the head supporting portion includes a generally u-shaped head supporting member configured to support a user's head when the user lies adjacent the shoulder support.

10. The orthopedic shoulder support of claim 9, wherein the head supporting portion further includes an attachment member extending outwardly from a head portion of the body, wherein the head supporting member is attached to a distal end of the attachment member relative to the body.

11. The orthopedic shoulder support of claim 6, wherein:
the body is a first open cell polyurethane foam having a first indentation load deflection;
the shoulder support surface pad is a second open cell polyurethane foam having a second indentation load deflection lower than the first indentation load deflection; and
and the arm support surface pad is a third open cell polyurethane foam having a third indentation load deflection lower than the first indentation load deflection.

12. An orthopedic pillow configured to stabilize a user's arm and shoulder while the user is sleeping adjacent the pillow on a support surface, the pillow comprising:
a generally horizontal shoulder support surface configured to be vertically spaced from the support surface when the orthopedic pillow is positioned on the support surface;
an upwardly sloping arm support surface extending from the shoulder support surface;
a generally vertical side wall extending from a side of the arm support surface and a side of the shoulder support surface; and
a flexible retention member attached to the pillow and configured to retain the pillow against a user's body.

13. The pillow of claim 12 wherein, the arm support surface has a first end proximate the shoulder support and a second end distal the shoulder support, wherein the second end is vertically spaced at a distance that is at least approximately equal to a distance from the support surface to a user's stomach when the user lies on the support surface.

14. The pillow of claim 12, wherein the side of the arm support surface is a first side of the arm support surface and the side of the shoulder support surface is a first side of the shoulder support surface, and further comprising:
a first generally vertical end wall extending from a second side of the shoulder support surface; and
a second generally vertical end wall extending from a second side of the arm support surface.

15. The pillow of claim 12 wherein the retention member is a strap configured to wrap around the user's body.

16. The pillow of claim 12 wherein the retention member extends outwardly from the pillow and is configured to support a user's weight when a user lies adjacent the pillow.

17. The pillow of claim 12 wherein the shoulder support surface, arm support surface, and sidewalls are formed from an open cell polyurethane foam with an indentation load deflection of about 100.

18. A method for manufacturing an orthopedic shoulder support for stabilizing a user's arm and shoulder, the method comprising:
forming a generally horizontal first surface positioned at a first distance above a bottom surface of the orthopedic shoulder support, wherein the first surface has length of about the distance from a top of a user's shoulder to a bottom of the user's shoulder; and
forming a second surface extending from the first surface at an upward angle relative to the bottom surface, the second surface having a proximal end portion positioned at the first distance above the bottom surface and a distal end portion positioned at a second distance above the bottom surface, the second distance being greater than the first distance;
wherein the second surface has a length of about a distance from the user's shoulder to a distal point of a user's elbow,
wherein the second surface has a width of about a maximum width of a user's upper arm, and
wherein the second distance is about a distance from the user's back to the distal point of the user's elbow; and
forming a third surface, wherein the third surface is a sidewall configured to extend upwardly from the first and second surfaces, and wherein the first, second, and third surfaces define a recess in the orthopedic shoulder support.

19. The method of claim 18, wherein:
forming the first surface comprises cutting a block of material;
forming the second surface comprises cutting the block of material; and
forming the third surface comprises cutting the block of material.

20. The method of claim 18, wherein forming the first surface comprises connecting a first portion of the orthopedic shoulder support to a second portion of the orthopedic shoulder support.
